(19) United States
(12) Patent Application Publication
(10) Pub. No.: US 2024/0033091 A1
(43) Pub. Date: Feb. 1, 2024

(54) A Flexible Osteochondral or Chondral Graft

(71) Applicant: Restoration Biologics LLC, Flagstaff, AZ (US)

(72) Inventors: Suzanne TABBAA, Flagstaff, AZ (US); Farshid GUILAK, Clayton, MO (US); William BUGBEE, San Diego, CA (US)

(21) Appl. No.: 18/378,765

(22) Filed: Oct. 11, 2023

Related U.S. Application Data
(63) Continuation-in-part of application No. PCT/US2023/011154, filed on Jan. 19, 2023.
(60) Provisional application No. 63/415,054, filed on Oct. 11, 2022.
(60) Provisional application No. 63/300,830, filed on Jan. 19, 2022.

(51) Int. Cl. A61F 2/30

(52) U.S. Cl. CPC A61F 2/30756; A61F 2002/30131; A61F 2002/30831; A61F 2002/30113

(57) ABSTRACT

A flexible osteochondral tissue graft or graft containing cartilage and underlying bone is provided with slits formed by water cutting. Various slit designs, or grooves enhance the flexibility of the graft and create a controlled open-pore structure to enhance cell infiltration and/or tissue integration.

### CROSS-REFERENCE TO RELATED APPLICATIONS

This application claims the benefit of U.S. Provisional Patent Application No. 63/415,054 filed Oct. 11, 2022, entitled “A Flexible Osteochondral or Chondral Graft” and this application is a continuation in part of International Application No. PCT/US2023/011154, filed on Jan. 19, 2023, entitled “Shaped Tissue Graft and Process to Maintain Properties” which claims the benefit of U.S. Provisional Patent Application No. 63/300,830, entitled “Patient Specific Graft and Process to Maintain Properties” and filed on Jan. 19, 2022, all of which are incorporated by reference.

### FIELD OF THE INVENTION

The present invention relates to tissue grafts and processing methods used for tissue repair.

### INCORPORATION BY REFERENCE

All publications and patent applications mentioned in this specification are herein incorporated by reference in their entirety to the same extent as if each individual publication or patent application was specifically and individually indicated to be incorporated by reference. Specifically, U.S. Pat. Nos. 10,251,751 and 10,722,370 and WO2023/141215 provide some background on tissue grafts and are incorporated herein by reference.

### BACKGROUND

Osteochondral allografting is a type of transplant procedure employing tissue grafts used to treat diseased individuals by harvesting a graft from a doner site in an individual and placing the graft in an implant site in a patient. Osteochondral or chondral tissue grafts are sourced from tissues derived from human or animals (e.g. allograft, xenograft, autograft). Cartilage is a smooth tissue that covers the ends of bones that meet at the joints and allows the bones to glide over each other with very little friction. Damaged cartilage does not heal easily. Osteochondral allografting is a type of cartilage transplant procedure used to treat individuals with cartilage injury or disease. The procedure involves transplanting a piece of articular cartilage and attached subchondral bone to a damaged section of the articular surface of a joint.

Tissue grafts are processed using various physical techniques to cut, shape, separate layers, or modify the architecture of the graft to generate a specific form for a clinical application. For example, U.S. Pat. No. 10,251,751 B2 describes traditional methods to cut grooves in osteochondral grafts. The grooves are cut completely through the bone and allow bending only at the cartilage layer. U.S. Pat. No. 10,722,370 B2 also describes traditional methods to cut grooves in osteochondral grafts.

The implant site can be an osteochondral or chondral defect in a human or veterinary patient. The implant site is specific to the joint and the location of the chondral or osteochondral defect within the joint. Osteochondral allografting can provide viable or devitalized cartilage and supporting bone that will be sufficient to maintain joint function and thereby relieve pain and reduce further damage to the articulation. Even though issue grafts include hard or soft tissues from various sources including human or animals (e.g. allograft, xenograft, autograft); unfortunately, there is a limited number of osteochondral grafts and often the grafts do not fit the implant site. Therefore, there is a need for grafts that can more easily fit different shaped implant sites.

### SUMMARY

A flexible graft such as an osteochondral tissue graft, or a graft containing cartilage and underlying bone or a chondral tissue graft containing only cartilage and/or a thin layer of underlying bone includes a modification of the bone layer and/or cartilage layers and/or transition layers (calcified cartilage region) with various, slit designs, or grooves to enhance the flexibility of the graft and create a controlled open-pore structure to enhance cell infiltration and/or tissue integration.

One preferred embodiment is directed to a flexible osteochondral graft for implantation into an implant site. The implant site is formed in a certain shape having a radius of curvature. The graft includes a cartilage layer. The graft also includes a boney region attached to the cartilage layer and an outer surface. When the graft is first removed from a doner, the graft is in a harvested state with a first radius of curvature. The graft is then modified to be in an implantation state. In the implantation state the outer surface of the boney region is provided with a plurality of linear cuts that serve to decrease the radius of curvature of the outer surface from the first radius of curvature in the harvested state to a second radius of curvature configured to complement the radius of curvature of the implant site. Preferably the plurality of linear cuts further includes a first set of linear cuts extending from the outer surface of the boney region to a first predetermined depth, a second set of linear cuts within the boney region extending from a location between the outer surface and the first predetermined depth to a second predetermined depth, and a third set of linear cuts within the boney region extending from a location between the first predetermined depth and the second predetermined depth and extending substantially through an opposing boney surface. Several dimensions of the cuts are preferable. For example, the linear cuts preferably extend at least 50% into the boney region and have a width between 0.01 mm and 1 mm. The linear cuts are defined by a Cartesian coordinate system or by a polar coordinate system. The flexible osteochondral graft preferably includes a protrusion extending from the outer surface of the boney portion. The cartilage layer and the boney region collectively form a substantially round, oblong, oval, rectangular, or irregular shape. The cartilage layer further comprises a surface and at least three distinct boney portions protruding from the surface.

The different cuts facilitate the infiltration of external biologics (e.g. bone marrow aspirate, platelet-rich plasma, blood, stem cells, etc.) prior to implantation. This invention also describes the method for producing a modified flexible osteochondral or chondral graft with specific and precisely-designed slits to allow shaping of the graft in multiple dimensions simultaneously. The method involves precise cutting of the osteochondral and/or chondral tissue with a waterjet or water cutter system to minimize tissue damage and preserve an open-pore architecture to allow tissue integration. A water-cutter involves the use of a high-pressure/high-velocity water jet, with or without a particulate abrasive additive (degradable or non-degradable) as an enhancer. The method can involve the use of biocompatible, non-degradable, degradable, and/or particulates to enhance the water-cutter technique.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a schematic view of a water cutter for cutting tissue grafts.

FIG. 2 shows processing steps to generate flexible tissue grafts.

FIGS. 3 and 4 shows a tissue graft cut with water-cutting.

FIGS. 5 and 6 show a tissue graft cut with traditional power tool techniques.

FIG. 7 shows a tissue graft cut with water-cutting.

FIG. 8 shows a tissue graft cut with traditional power tool techniques.

FIG. 9 shows cartilage tissue cut with a waterjet contains more viable cells than tradition cutting.

FIG. 10 shows cartilage tissue cut with traditional cutting tools.

FIG. 11 is a perspective view of an osteochondral graft with a simple slit design.

FIGS. 12, 13, and 14 are perspective views of osteochondral or chondral grafts with lattice patterns to allow for flexibility, integration and cellular infiltration.

FIG. 15 is a perspective view of a chondral structure with slits through deep and middle layers.

FIGS. 16, 17, and 18, are perspective views chondral structures with or without minimal subchondral bone containing pores that transverse the bone and deeper layers of the cartilage but does not penetrate the superficial cartilage.

FIGS. 19, 20, and 21 are views of an oblong shaped osteochondral graft with slits to allow for flexibility and matching of recipient site radius of curvature.

FIG. 22 shows a chondral graft with small pieces of subchondral bone or calcified cartilage remaining in the shape of pegs.

FIGS. 23 and 24 show top and bottom perspective views of a chondral graft with a lattice structure cut at two angles to allow bendability in two directions to match curvature.

FIG. 25 shows a perspective view of a graft with linear pattern cuts to allow flexibility and a single peg for enhanced integration and fixation.

FIG. 26 shows a perspective view of a graft with linear pattern cuts to allow flexibility and multiple pegs for enhanced integration and fixation opportunities.

FIG. 27 shows a perspective view of a graft with linear pattern cuts to allow flexibility and recess cuts in hard tissue for suture placement beneath cartilage.

### DETAILED DESCRIPTION

The following detailed description should be read with reference to the drawings in which similar elements in different drawings are numbered the same. The detailed description and the drawings, which are not necessarily to scale, set forth illustrative and exemplary embodiments and are not intended to limit the scope of the disclosure. Selected features of any illustrative embodiment can be incorporated into an additional embodiment unless clearly stated to the contrary. While the disclosure is amenable to various modifications and alternative forms, specifics thereof have been shown by way of example in the drawings and will be described in detail. As used herein in the specification and claims, including as used in the examples and unless otherwise expressly specified, all numbers may be read as if prefaced by the word “about” or “approximately,” even if the term does not expressly appear, with it being understood that this provides a reasonable expected range of values in the order of +/−10% of the stated value (or range of values). In addition, any numerical range recited herein is intended to include all sub-ranges subsumed therein. Overall, it should be understood, however, that the intention is not to limit aspects of the disclosure to the particular illustrative embodiments described. On the contrary, the intention is to cover all modifications, equivalents, and alternatives falling within the spirit and scope of the disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Embodiments of aspects of the present invention relate to producing a flexible osteochondral/chondral graft. Osteochondral or chondral tissue grafts are sourced from any tissues derived from human or animals (e.g. allograft, xenograft, autograft). The recipient or implant site can be an osteochondral or chondral defect from a human or veterinary patient. The implant site is specific to the joint and the location of the chondral or osteochondral defect within the joint. One embodiment of this invention involves creating flexible osteochondral or chondral tissues using a kerfing technique and water cutter system based on the radius of curvature of the implant site.

### Water Cutter System Used to Produce Flexible Graft

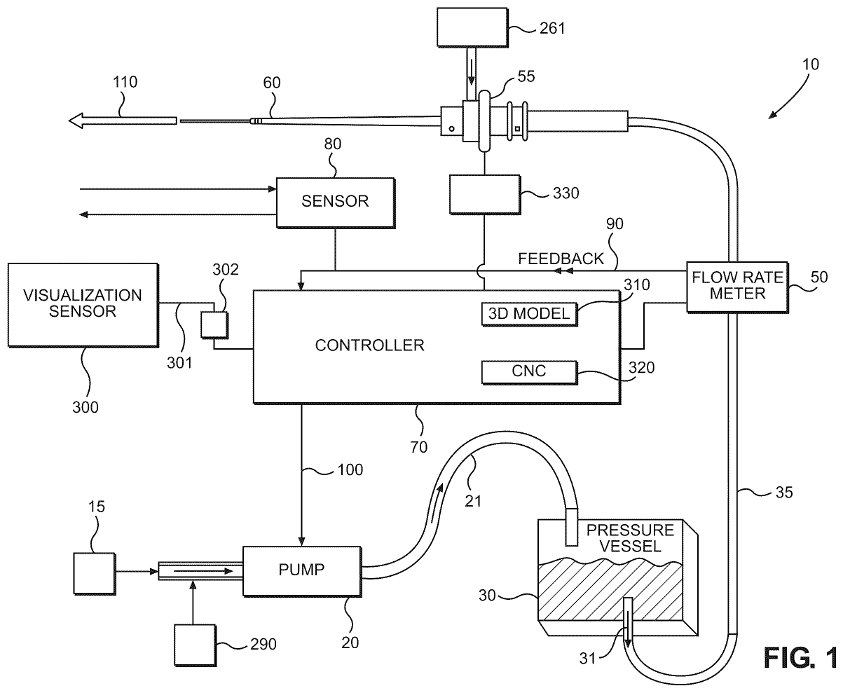

Referring to FIG. 1, there is shown a water cutter system 10 for creating flexible osteochondral of chondral tissue grafts. In use, fluid from a water source 15 enters a pump 20 and is forwarded to a pressure vessel 30 via a pump output tube 21. The fluid travels through output 31 of pressure vessel 30 and is forwarded along a transfer tube 35 to the flow rate meter 50. An output fluid from flow rate meter 50 travels to a manifold 55 and then to a nozzle 60. An adjustment of the diameter of nozzle 60 is controlled by feedback to controller 70 from a depth sensor 80 along loop 90 and then to pump 20 along communications channel 100. Controller 70, cut depth sensor 80 and pump 20 work together to control cutting grafts. Controller 70 may be a computer, a central processing unit, a microcontroller, ASIC, or other control circuitry. Fluid flow at flow rate meter 50 is controlled throughout the cut of a particular section of bone in the graft. More particularly, cutting area, shape of a jet 110, and pressure, for example, of the cutting fluid can be precisely controlled via the continuous feedback.

Advantageously, cutting pressure can be controlled via the feedback loop 90, communications channel 100 and adjustable nozzle 60 such that flow rate meter 50 outputs a minimum pressure sufficient to cut a particular section of bone in order to minimize damage to the surrounding soft tissue. As bone is heterogeneous, the flow rate can be continuously adjusted to maintain minimum sufficient pressure throughout the depth of the cut as different density of bone is encountered.

Fluid flow, including fluid pressure and the length of time and the pressure that must be applied to a particular cutting region to make a cut of a certain depth can be preoperatively determined from the bone quality data. However, water cutter system 10 may optionally further include sensor 80 for determining a cut depth. Sensor 80 may be, for example, an ultrasonic sensor or an optical sensor for verifying the cut depth. A signal may be sent from sensor 80 and reflected off of the cutting region of the bone such that real-time cutting depth information can be transmitted to controller 70 to verify accuracy and/or adjust the fluid flow, if necessary.

The water cutter system 10 preferably used combination with a particulate abrasive additive (degradable or non-degradable) as an enhancer to create the slit or cuts in the osteochondral tissue. The additive is preferably added to from a source 261 to manifold 55 so that the additive is incorporated into a water jet 110. The additive involves biocompatible and biodegradable materials which could include but is not limited (e.g. sodium chloride, calcium sulfate, calcium phosphate, etc.), sugar crystals (sucrose, glucose, etc.), calcium carbonate, biodegradable polymers, tissue particle (e.g. bone or cartilage particles), ice or dry ice particles, and other biomaterials. The particulate biomaterial properties (size, chemistry, architecture) used for the abrasive is tailored to various applications. The particulate is used to enhance the cutting efficiency and to modify the tissue roughness and micro-architecture. In another embodiment, water cutter system 10 uses physiological buffered solutions such as phosphate buffered saline or solutions of controlled osmolarity (e.g., hyperosmotic solution) to maintain viability and biological properties of the tissue. In another embodiment, the water cutter solution may involve acids, enzymes, or other chemicals that can demineralize the surface of the bone or modify the architecture. In one example, the sugar particles are sucrose with particle sizes that range from 10-800 μm. In another example, salt crystals, powder, or granules with varying dissolution rates of particle sizes that ranges from 10-800 μm. Another ideal range of the salt particle may include 100-600 μm or 200-400 μm. Examples include sodium chloride, zinc oxide, magnesium oxide, magnesium sulfate, zinc acetate, zinc sulfate. Bone particles derived from human or animal bone may be used as an abrasive. These particles could range from 10-800 μm. Synthetic bone particles derived from calcium phosphate may also be used as an abrasive. These particles could vary in size, dissolution rate, and shape. Irregular calcium phosphate particle or granules of sizes between may be used to generate roughened surfaces or surfaces with more surface area to promote osseointegration. Such solutions are preferably added from a source 290 before the pump but could be added in other places. The source 290 is preferably employed to add numerous different components to the water supplied at water source 15. In another embodiment, the water cutter system 10 uses cell-compatible solutes such as salts or sugars to alter the osmolarity of the water cutting solution to maintain viability and biological properties of the tissue. In another embodiment, the water cutter solution involves acids or other chemicals that can demineralize the surface of the bone or modify the architecture. In another embodiment, the water cutter solution includes drugs or enzymes to stimulate or enhance biological properties. A visualization system 300 may be provided to send along a communications channel 301, data 302 such as 2D or 3D images of the implant site and patient anatomy to controller 70. Data 302 can be used to form a 3D model 310 used by a CNC program 320.

### Method of Producing Flexible Grafts

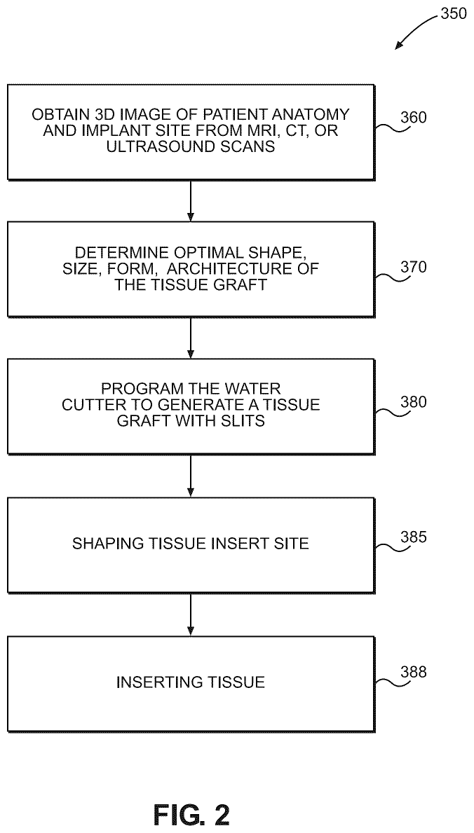

In one embodiment, an osteochondral, chondral, or bone flexible tissue graft is formed using water cutter system 10. A method 350 of producing flexible tissue grafts is shown in FIG. 2 and includes, at 360, obtaining 2D or 3D images of the implant site and patient anatomy. 2D or 3D images, represented by data 302, could be obtained with the visualization system 300, using various imaging technologies including x-ray (radiographs), computed topography (CT), ultrasound, or magnetic resonance imaging (MRI). The desired shape, size, and architecture of the tissue is determined by the medical imaging scan. Preferably, a 3D image of a patient's targeted implant site and surrounding anatomy is obtained. A boundary of the implant site is obtained and noted. The scan is preferably used for computer-aided processing of an osteochondral graft by water cutter system 10.

Computer systems and software programming tools are preferably used, at 370, to determine a 3D model of the tissue graft with exact shape, form, size, and architecture based on the dimensions and shape of the implant site or defect identified by the 3D images of the patient acquired from MM. A 3D model of a graft is created by utilizing the boundary of the implant site, a surface curvature, extrapolated from the patient's target implant site surrounding anatomy, and a depth, defined by a plane parallel to a plane normal to a point of curvature at the surface. A donor tissue is chosen that has a location with a close approximation to the 3D model of the flexible graft. The donor tissue is visualized to determine a cutting path to shape or modify an architecture of the tissue.

Water cutter system 10 is preferably programmed, at 380, to form the exact size and shape of the desired flexible graft including slits. Preferably forming the modified tissue architecture includes shaping or bending of a tissue graft in one or multiple axes to match a prescribe shape or curvature using controlled removal of slits, pores, or other prescribed regions of the tissue. A cutting path is identified with imaging technology and computational methods. The graft is cut out of the donor tissue utilizing a fluid cutting process with computer numerical controls to cut the graft from the donor tissue along the cutting path. The processing parameters of the fluid cutting system, at 370 and 380, directly impact the remaining surface of the cut tissue. Embodiments of the present invention impact the tissue micro-architecture, roughness, nanoroughness, density, surface energy, surface charge, as well as other parameters through modifications of the processing parameters. Examples of such parameters include fluid media, abrasive media, pressure, flow rate, nozzle diameter, cutting distance, cutting speed, dwell time, and processing time. The combination of parameters depends on the type of tissue being cut (e.g. bone, cartilage, skin, etc.) as well as the desired surface properties after cutting. The speed of cutting may range from low to very high speeds depending on the surface roughness desired. The speed may vary from 0.01 inches per minute to 10 inches per minute.

The recipient site needs to be prepared, at 385, to remove tissue such that it can be replaced with the graft 230, at 388. Preferably, shaping a tissue bed or removing autologous patient tissue in vivo during a surgical procedure, includes preparing a surgical site in a proper geometry removing tissues in a site-specific manner, to remove necrotic tissue, tumors, burns, or fibrous tissue; and removing foreign bodies or particles from native tissue, such as dirt, gravel, tattoo ink, projectiles, etc. In one example, the recipient site involves skin or dermal tissue and the fluid cutter system is used for debridement or dermabrasion.

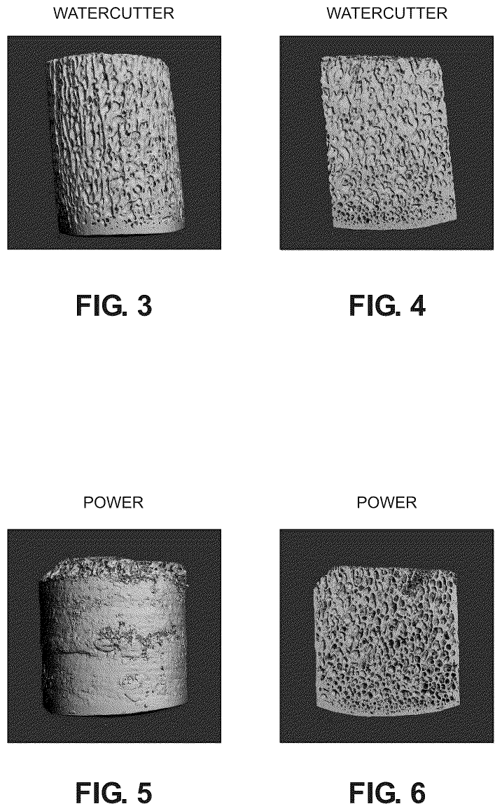

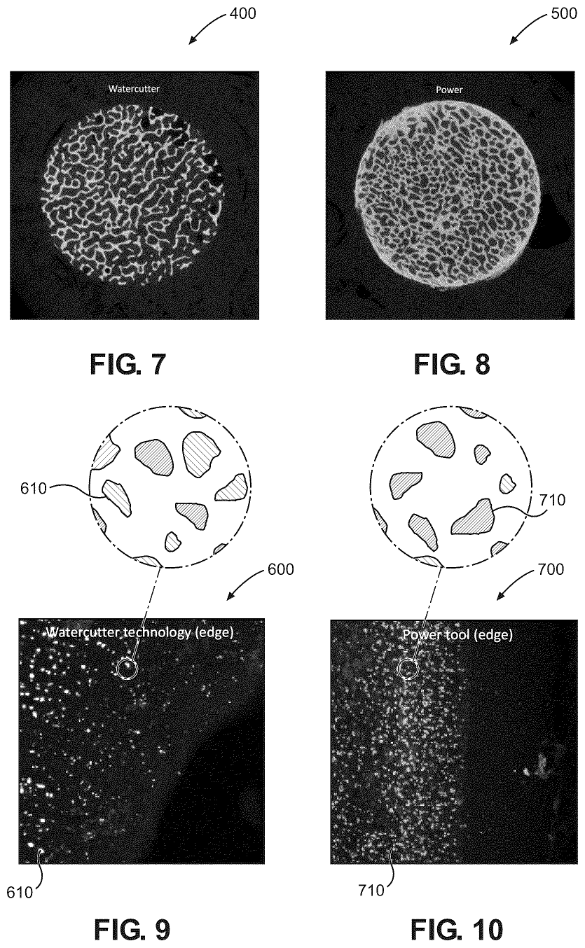

Employing water cutting system 10 yields better grafts when compared to those produced by power tools. See for example, the grafts shown in FIGS. 3 and 4 cut with water cutters versus the grafts shown in FIGS. 5 and 6 cut with power tools. See also graft 400 in FIG. 7 cut with a water cutter versus the graft 500 of FIG. 8 cut with conventional cutters. Note also, FIG. 9 shows cartilage tissue 600 adjacent a cut edge formed with a waterjet which contains more viable cells 610 than an edge formed with tradition cutting. FIG. 10 shows cartilage tissue 700 adjacent to an edge cut with traditional cutting methods. The edge has increased levels of dead cells 710. Cell viability has been shown to be an important factor in graft integration and longevity in vivo.

### Flexible Osteochondral Graft Design

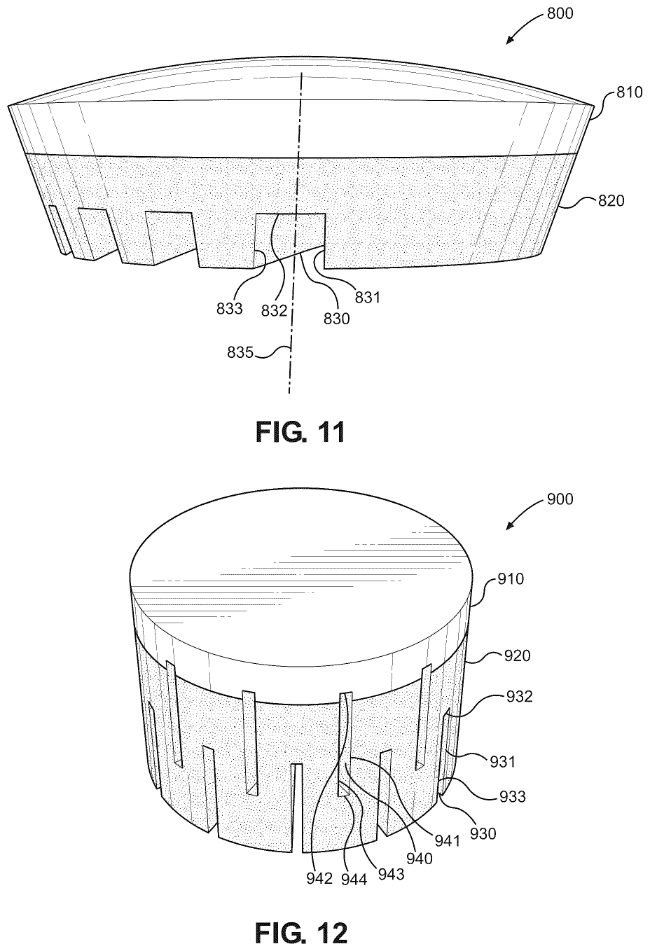

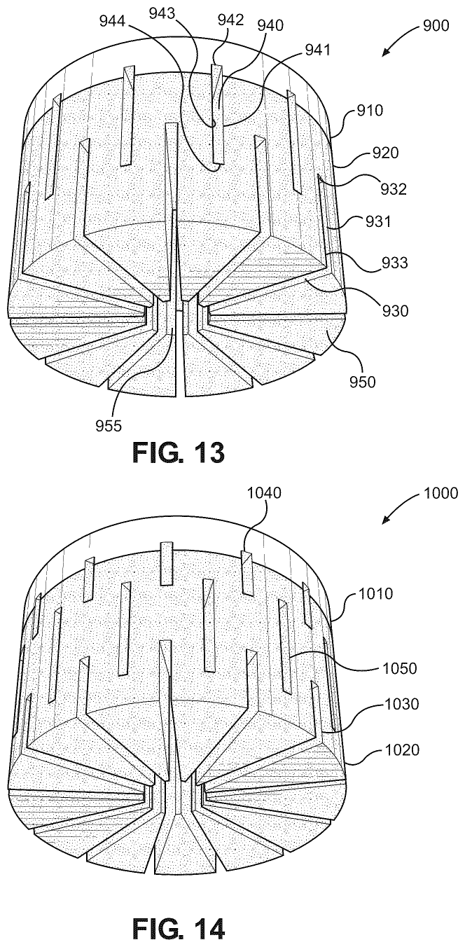

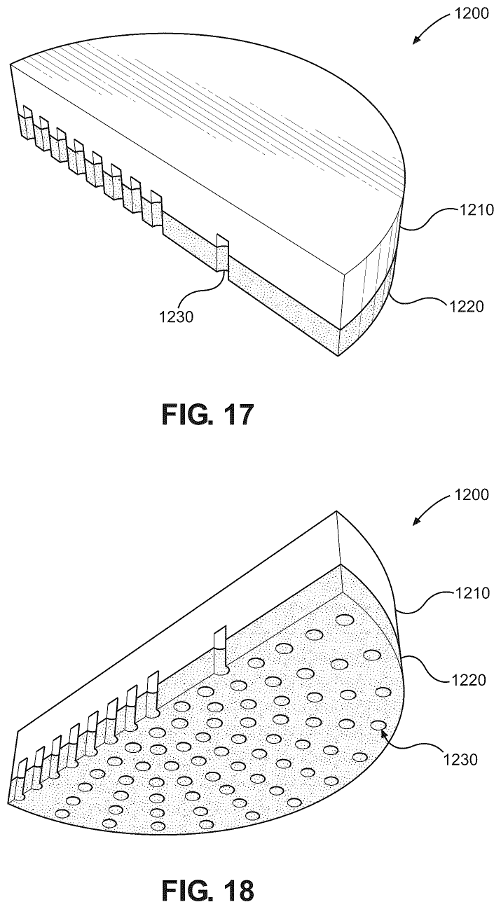

According to yet another preferred embodiment of the invention, as seen FIGS. 11-18, an osteochondral or chondral graft with various slit or pore designs or patterns formed by water cutting produces a flexible graft or a graft that can match the radius of curvature of the recipient site in multiple axes. FIG. 11 shows a flexible graft 800 shown in an implantation state with a cartilage layer 810 having an average thickness of at least 0.5 mm and an outer surface. Cartilage layer 810 is connected to a boney region or layer 820. Graft 800 has a simple serial slit design. Note slit 830 having a side wall 831, an end wall 832 and an opposing side wall 833. Graft 800 has a radius of curvature 835 of the outer surface that has been decreased from a harvested state to an implantation state by the size and placement of the cuts or slits. Radius of curvature 835 is configured to complement the implantation site radius of curvature. FIGS. 12 and 13 show a graft 900 with a cartilage layer 910 and a boney region or layer 920 with two sets or pluralities of linear cuts or slits. Cartilage layer 910 and boney layer 920 form an interface or opposing boney surface. The first set of slits has slits similar to slit 830. Note slit 930 having a side wall 931, an end wall 932 and an opposing sidewall 933. The second set of slits pass through subchondral bone, penetrating the calcified cartilage. Note slit 940 having a side wall 941 and end wall 942 formed in the cartilage layer 910 and opposing side wall 943. In addition, slit 940 has a bottom wall 944 since slit 940 does not extend to an outer surface 950 of graft 900. Surface 950 has a central opening 955 were the slits meet. Slit 930 extends a predetermined depth from surface 950 to end wall 932 which is at least 50% into boney region 920. Preferably slit 930 has a width of between 0.01 mm and 1 mm as defined by a Cartesian coordinate system or a polar coordinate system. Slit 940 starts at bottom wall 944 which is a predetermined distance from surface 950 such that bottom wall 944 is between bottom surface 950 and end wall 932. Slit 940 extends another predetermined distance from bottom wall 944 to end wall 942 and preferably terminates within cartilage layer 910 after passing substantially through the opposing boney surface.

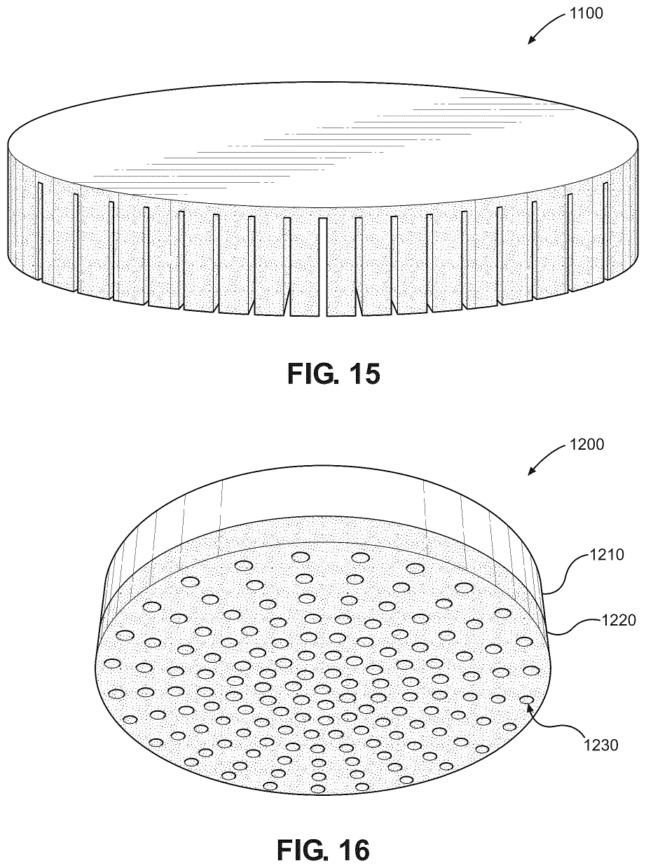

FIG. 14 shows another flexible graft 1000 having a cartilage layer 1010 and a boney layer 1020 with a set of slits with a slit 1030 similar to slit 930, a slit 1040 similar to slit 940 and an intermediate set of slits. See slit 1050 for example. FIG. 15 shows a graft 1100 having a chondral structure with slits through deep and middle layers. FIGS. 16-18 show a graft 1200 with a cartilage layer 1210 and a boney region or layer 1220 with a series of pores or circular cuts 1230 which extend at least 50% into the boney region 1220 have a diameter of between 0.01 mm and 1 mm. The circular cuts 1230 are defined by a Cartesian coordinate system or a polar coordinate system. The chondral structure with sub chondral bone contains pores 1230. Pores 1230 transverse the bone and deeper layers of cartilage but not the superficial cartilage.

The slit patterns could include serial slits that transverse the bony section and the entire depth of the bone. The slits could also penetrate both the calcified cartilage layer and the bone. Other designs include a slit or cut patterns that result in a lattice hinge, linear pattern design, or other patterns such as osteochondral or chondral grafts with radial lattice patterns.

Any osteochondral or chondral tissue from animal or human joints can be used to generate a flexible graft or a graft with flexibility to match the ROC of a recipient implant site. The osteochondral tissue with slit patterns retains both tissue viability and architecture. Specifically, the peripheral tissue near the cuts maintains the native tissue architecture. In one aspect, an osteochondral graft with slits or cuts in a specific pattern is produced to provide a flexible graft. In another aspect, the principal radii of curvature (ROC) of the recipient site are measured using imaging and used to determine the pattern type and slit design of the osteochondral graft and the architecture of the tissue to enhance the biological properties and ability of the tissue to integrate. The flexible osteochondral graft is produced using the slit design or pattern.

The pattern, number of slits, size of slits, density of slits per region, and length can be modified to produce grafts of various radius of curvature and flexibility. The pattern and slit design may vary between the cartilage zones and underlying bone. These variables are modified based on the recipient-site ROC or flexibility needs.

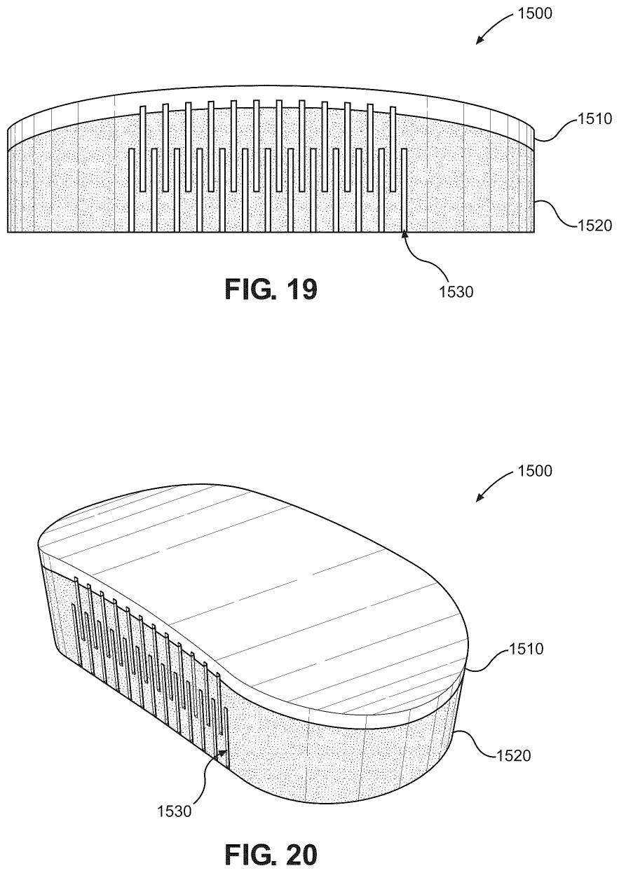

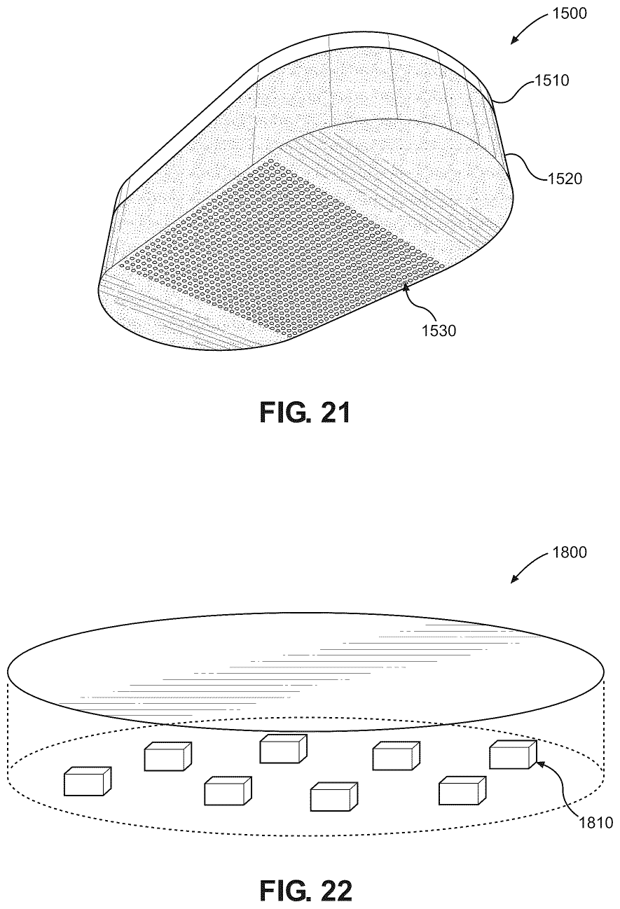

In another embodiment, the osteochondral or chondral graft may have a cylindrical form with slits only in specific regions to obtain a ROC. The osteochondral or chondral graft 1500 may have a rectangular or oblong shape as best seen in FIGS. 19-21. Graft 1500 has a cartilage layer 1510 and a boney region 1520 with slits 1530 throughout the width or in specific regions to provide areas of flexibility. Cartilage layer 1510 and boney region 1520 collectively form a substantially round, oblong, oval, rectangular, or irregular shape. Flexible oblong shape osteochondral graft with slits to increase biologic augmentation (e.g. platelet rich plasma or bone marrow aspirate) and improve tissue integration.

In another embodiment, best seen in FIG. 22. Chondral graft 1800 is provided with small pieces of subchondral bone or calcified cartilage remaining in the shape of pegs 1810, the chondral graft may have only small, predefined regions of bone or calcified cartilage left remaining to allow for attachment and integration to the graft site. In one example, not shown, the osteochondral graft may include pegs of various shapes including but not limited to round, cruciform, square, or bullet shape. The graft may include one to twelve pegs. In one example, the osteochondral graft is oblong shape and contains 4 pegs of round or cruciform shape to reduce micromotion or toggling of the graft.

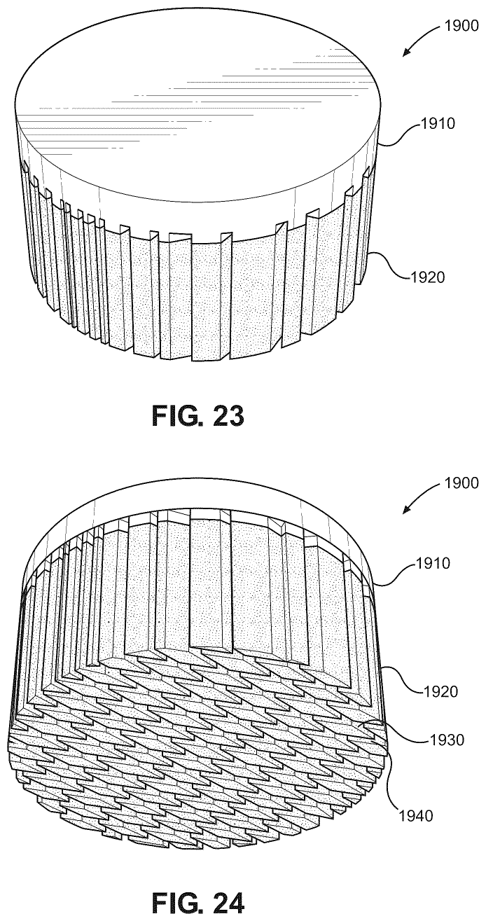

In another embodiment, FIGS. 23 and 24, a graft 1900 is shown with a cartilage layer 1910 and a boney layer 1920. Boney layer 1920 is formed with cuts 1930 and 1940 to form a lattice structure cut at two angles to allow bendability in two directions to match curvature. Slits or cuts 1930, 1940, are made in multiple directions or axes (orthogonal or offset to one another) to allow bending and formation of the specific graft curvatures in two axes in a predefined manner.

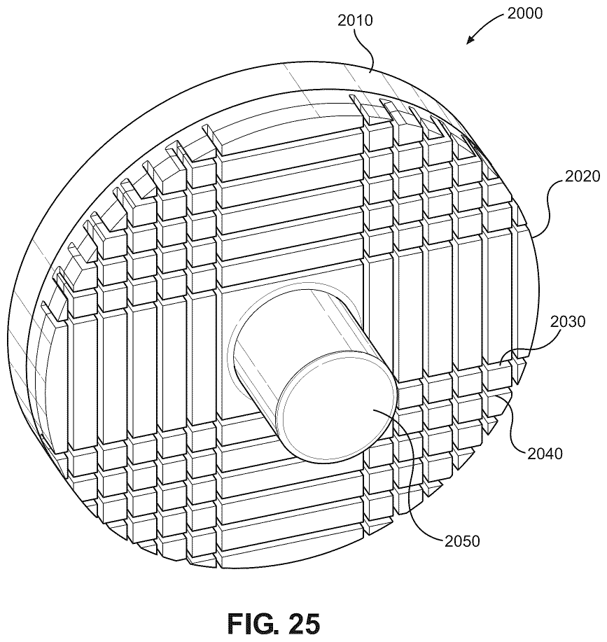

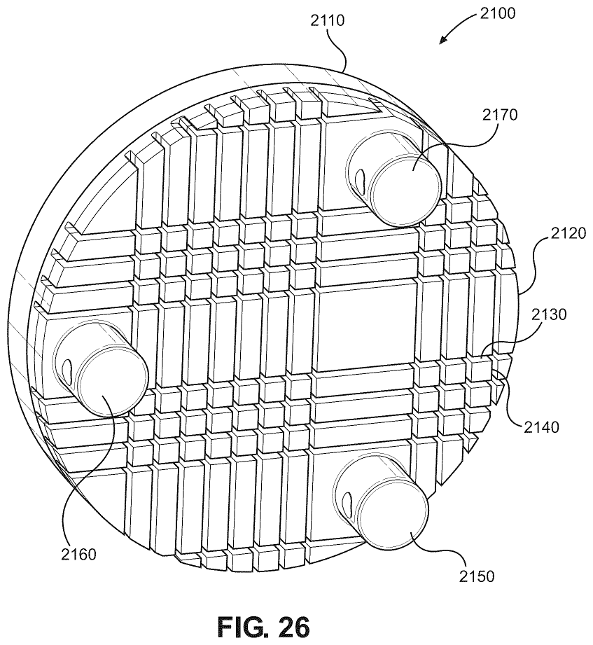

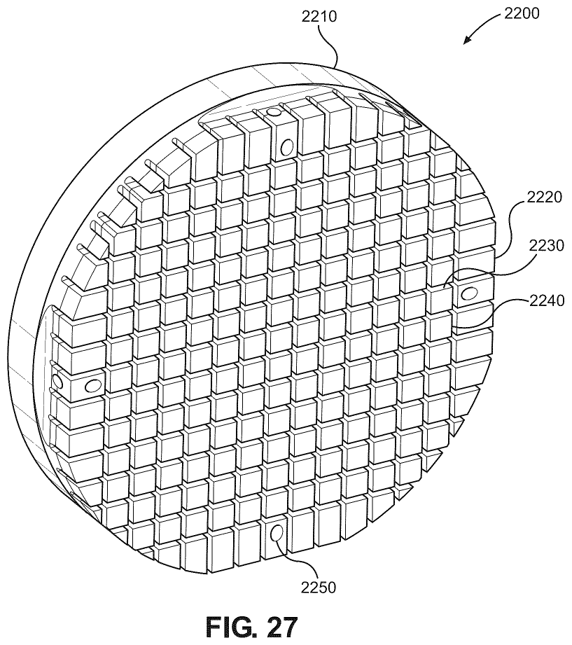

In another embodiment, as shown in FIG. 25, a graft 2000 is formed with a cartilage layer 2010 and a boney region or layer 2020. Slits 2030 run in a first direction and slits 2040 run in a second direction to form slit patterns. A protrusion or peg 2050 protrudes from an outer surface of boney layer 2020 of graft 2000. FIG. 26 shows a graft 2100 also formed with a cartilage layer 2110 and a boney layer 2120. Once again slits 2130 run in a first direction and slits 2140 run in a second direction to form slit patterns. This time three distinct boney regions or pegs 2150, 2160, and 2170 are formed. The pegs and slit patterns are combined in the same graft to achieve flexibility to obtain a ROC and enhance integration to the graft site. FIG. 27 shows a graft 2200 with a cartilage layer 2210 and a boney layer 2220. Once again slits 2230 run in a first direction and slits 2240 run in a second direction. A recess 2250 is formed to accept stitches to secured graft 2200 in an implant site.

Although various illustrative embodiments are described above, any of a number of changes may be made to various embodiments without departing from the scope of the invention as described by the claims. For example, any of the above described modified osteochondral or chondral grafts may be combined with another material or biologics such as autologous bone marrow aspirate, platelet-rich plasma, stem cells, growth factors, etc. In addition, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

1. A flexible osteochondral graft for implantation into a implantation site having a implantation site radius of curvature said graft comprising:
a cartilage layer and
a boney region attached to the cartilage layer and having an outer surface wherein the graft has a harvested state with a first radius of curvature and an implantation state wherein an outer surface of the boney region is provided with a plurality of linear cuts that serve to decrease the radius of curvature of the outer surface from the first radius of curvature in the harvested state to a second radius of curvature configured to complement the implantation site radius of curvature.

2. The flexible osteochondral graft of claim 1, wherein the plurality of linear cuts includes a first set of linear cuts extending from the outer surface of the boney region to a predetermined depth in the boney region and a second set of linear cuts within the boney region extending from a location between the surface on the predetermined depth of the first set and substantially through an opposing boney surface.

3. The osteochondral graft of claim 1, wherein the plurality of linear cuts includes a first set of linear cuts extending from the outer surface of the boney region to a first predetermined depth, a second set of linear cuts within the boney region extending from a location between the outer surface and the first predetermined depth to a second predetermined depth, and a third set of linear cuts within the boney region extending from a location between the first predetermined depth and the second predetermined depth and extending substantially through an opposing boney surface.

4. The flexible osteochondral graft of claim 1, wherein the linear cuts extend at least 50% into the boney region.

5. The flexible osteochondral graft of claim 1, wherein the linear cuts have a width between 0.01 mm and 1 mm.

6. The flexible osteochondral graft of claim 1, wherein the linear cuts are defined by a Cartesian coordinate system.

7. The osteochondral graft of claim 1, wherein the linear cuts are defined by a polar coordinate system.

8. The flexible osteochondral graft of claim 1, further comprising a protrusion extending from the outer surface of the boney region.

9. The flexible osteochondral graft of claim 1 wherein the cartilage layer and the boney region collectively form a substantially round, oblong, oval, rectangular, or irregular shape.

10. The flexible osteochondral graft of claim 1, wherein the cartilage layer further comprises a surface and at least three distinct boney portions protruding from the surface.

11. A flexible osteochondral graft for implantation into an implant site having a radius of curvature said graft comprising:
a cartilage layer and
a boney region attached to the cartilage layer and having an outer surface wherein the graft has a harvested state with a first radius of curvature and an implantation state wherein an outer surface of the boney region is provided with a plurality of circular cuts that serve to decrease the radius of curvature of the outer surface from the first radius of curvature in the harvested state to a second radius of curvature configured to complement the radius of curvature of the implant site.

12. The flexible osteochondral graft of claim 11, wherein the circular cuts extend at least 50% into the boney region.

13. The flexible osteochondral graft of claim 11, wherein the circular cuts have a diameter between 0.01 mm and 1 mm.

14. The flexible osteochondral graft of claim 11, wherein the circular cuts are defined by a Cartesian coordinate system.

15. The flexible osteochondral graft of claim 11, wherein the circular cuts are defined by a polar coordinate system.

16. The flexible osteochondral graft of claim 11, further comprising a protrusion extending from the outer surface of the boney region.

17. The flexible osteochondral graft of claim 11, wherein the cartilage layer and the boney region collectively form a substantially round, oblong, oval, rectangular, or irregular shape.

18. The flexible osteochondral graft of claim 11, wherein the cartilage layer further comprises a surface and at least three distinct boney portions protrude from the surface.

19. A flexible osteochondral graft for implantation into an implant site having a radius of curvature said graft comprising:
a cartilage layer and
a boney region attached to the cartilage layer and having an outer surface wherein the graft has a harvested state with a first radius of curvature and an implantation state wherein an outer surface of the boney region is provided with a plurality of linear cuts formed by waterjet cutting that serve to decrease the radius of curvature of the outer surface from the first radius of curvature in the harvested state to a second radius of curvature configured to complement the radius of curvature of the implant site, wherein the plurality of cuts includes
a first set of cuts extending from the outer surface of the boney region to a predetermined depth and a second set of linear cuts within the boney region extending from a location between the surface on the predetermined depth of the first set of cuts and substantially through an opposing boney surface; wherein the cuts are between 0.01 mm and 1 mm in width; and wherein the cartilage layer has an average thickness of at least 0.5 mm.
